(19) United States
(12) Patent Application Publication
(10) Pub. No.: US 2014/0349357 A1
(43) Pub. Date: Nov. 27, 2014

(54) METHODS OF PRODUCING BUTANOL FROM NON-CELLULOSIC BIOMASS

(71) Applicant: 3M INNOVATIVE PROPERTIES COMPANY, St. Paul, MN (US)

(72) Inventors: Masayuki Nakamura, Woodbury, MN (US); Dan L. Fanselow, White Bear Lake, MN (US); Michaela Kathleen Culhane, St. Paul, MN (US)

(73) Assignee: 3M Innovative Properties Company, St. Paul, MN (US)

(21) Appl. No.: 14/364,365

(22) PCT Filed: Dec. 4, 2012
(86) PCT No.: PCT/US2012/067755

§ 371 Date: Jun. 11, 2014

Related U.S. Application Data
(60) Provisional application No. 61/570,639, filed on Dec. 14, 2011.

(51) Int. Cl. C12P 7/16; C07C 29/86

(52) U.S. Cl. CPC C12P 7/16; C07C 29/86
USPC 435/160

(57) ABSTRACT

Methods of recovering butanol, and preferably increasing the rate and/or yield of its production, from the fermentation of material derived from the digestion of non-cellulosic biomass.

### CROSS REFERENCE TO RELATED APPLICATIONS

This application claims priority to U.S. Provisional Patent Application 61/570,639, filed on Dec. 14, 2011, the disclosure of which is incorporated by reference in their entirety.

### BACKGROUND

Non-cellulosic biomass is becoming an increasingly important source for fuel production in efforts to develop sustainable energy. Butanol is a high-value molecule as a building block chemical and a potential “drop-in” transportation fuel. Butanol has the following added advantages as a transportation fuel beyond those offered by ethanol, for its higher energy density and lower water uptake. Though two types of butanol (1-butanol and isobutanol) are currently produced from crude oil, butanols can also be produced via fermentation using corn-derived sugars and possibly cellulosic-derived sugars. However, butanol production from the fermentation processes on a large scale is hindered by technical and economic challenges.

One of these challenges is the low titer of butanol at the end of the fermentation process. Butanol is highly toxic to the microorganisms that form it. As a result, the final butanol concentration is often only 1-2 wt-%, compared to 12-20 wt-% ethanol in yeast fermentation broth. This reduces the recovery of butanol from fermentation broth. In addition, butanol has a higher boiling point (1-butanol at 117° C., isobutanol at 108° C.) than water (100° C.) making it difficult to separate economically by distillation. The low fermentation titer and higher boiling point of butanol result in significantly higher energy cost when standard distillation processes are used for butanol recovery, as described in the article by Kraemer et al., “Separation of butanol from acetone-butanol-ethanol fermentation by a hybrid extraction-distillation process,” Computer Aided Chemical Engineering, 28:7-12 (2010); in the article by Lee et al., “Fermentative Butanol Production by Clostridia,” Biotechnology and Bioengineering, 101:209-228 (2008); and in the article by Ezeji et al., “Bioproduction of Butanol from Biomass: from Genes to Bioreactors,” Current Opinion in Biotechnology, 18:220-227 (2007). To make biobutanol production economically viable, a cost effective recovery process is needed.

### SUMMARY

There is a continuing need for effective and efficient methods for obtaining fuels from non-cellulosic biomass. The present disclosure provides methods of recovering butanol, and preferably increasing the rate and/or yield of its production, from the fermentation of material derived from the digestion of non-cellulosic biomass. More specifically, in certain embodiments, the present disclosure provides butanol enrichment by membrane solvent extraction.

In one embodiment, a method of producing butanol includes: introducing an aqueous mixture comprising carbohydrates obtained from non-cellulosic biomass into a fermenter; fermenting the aqueous mixture to provide a first fermentation broth, the fermentation broth comprising: a microorganism for producing butanol; carbohydrates from the non-cellulosic biomass; and butanol; and at least partially extracting the butanol from the first fermentation broth with a first solvent extractant by a first liquid-liquid extraction through a first porous membrane to provide a first extract and a second fermentation broth. In this method the first solvent extractant comprises a straight-chain or branched alcohol having from 7 to 12 carbon atoms. As a result of this method, the second fermentation broth has a lower concentration of the butanol than the first fermentation broth. The first liquid-liquid extraction is carried out in a liquid-liquid extraction element comprising: a plurality of first layer pairs, each first layer pair comprising: a first polymeric microporous membrane; and a first flow channel layer oriented in a first flow direction having a first fluid inlet and a first fluid outlet disposed on first opposing sides of the liquid-liquid extraction element; and a plurality of second layer pairs, with at least one second layer pair being disposed between two first layer pairs and at least one first layer pair being disposed between two second layer pairs so as to form a stack of layers, each second layer pair comprising: a second polymeric microporous membrane; and a second flow channel layer oriented in a second flow direction different than the first flow direction and having a second fluid inlet and a second fluid outlet disposed on second opposing sides of the extraction element.

This method can increase the rate of butanol production by a factor or two or more when the secondary fermentation broth is directed back into the fermenter (compared to a method that does not do such recycling).

In one embodiment, a method of recovering butanol from a fermentation broth includes: introducing an aqueous mixture comprising carbohydrates obtained from non-cellulosic biomass into a fermenter; fermenting the aqueous mixture to provide a first fermentation broth, the fermentation broth comprising: a microorganism for producing butanol; carbohydrates from the non-cellulosic biomass; and butanol; at least partially extracting the butanol from the first fermentation broth with a first solvent extractant by a first liquid-liquid extraction through a first porous membrane to provide a first extract and a second fermentation broth; and recovering at least a portion of the butanol from the first extract. In this method the first solvent extractant comprises a straight-chain or branched alcohol having from 7 to 12 carbon atoms. As a result of this method, the second fermentation broth has a lower concentration of the butanol than the first fermentation broth. The first liquid-liquid extraction is carried out in a liquid-liquid extraction element comprising: a plurality of first layer pairs, each first layer pair comprising: a first polymeric microporous membrane; and a first flow channel layer oriented in a first flow direction having a first fluid inlet and a first fluid outlet disposed on first opposing sides of the liquid-liquid extraction element; and a plurality of second layer pairs, with at least one second layer pair being disposed between two first layer pairs and at least one first layer pair being disposed between two second layer pairs so as to form a stack of layers, each second layer pair comprising: a second polymeric microporous membrane; and a second flow channel layer oriented in a second flow direction different than the first flow direction and having a second fluid inlet and a second fluid outlet disposed on second opposing sides of the extraction element. Preferably, recovering the butanol comprises concentrating it by flash separation and/or vacuum distillation from the extraction solvent.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list. All numerical ranges are inclusive of their endpoints and non-integral values between the endpoints unless otherwise stated.

As used herein, the term “or” is generally employed in its usual sense including “and/or” unless the content clearly dictates otherwise. The term “and/or” means one or all of the listed elements or a combination of any two or more of the listed elements

The terms “first” and “second” are used in this disclosure. It will be understood that, unless otherwise noted, those terms are used in their relative sense only. In particular, in some embodiments certain components may be present in interchangeable and/or identical multiples (e.g., pairs). For these components, the designation of “first” and “second” may be applied to the components merely as a matter of convenience in the description of one or more of the embodiments.

The term “aqueous” refers to comprising water.

The term “butanol” refers either to 1-butanol or isobutanol, depending on the microorganism used in the fermentation process, or to a mixture of 1-butanol and isobutanol if a mixture of microorganisms is used.

The “extractant,” including the first extractant or second extractant includes one compound or a mixture of compounds. Typically the extractant refers to an organic solvent or a mixture of organic solvents.

“Liquid-liquid extraction” is a method for transferring a solute dissolved in a first liquid to a second liquid.

The term “entrained” includes when the first extractant is suspended, trapped, or dissolved in the aqueous mixture.

“Non-cellulosic biomass” means carbohydrates or materials containing greater than 1 percent by weight (wt-%) starch, dextrin, sugars (e.g., dextrose, sucrose, xylose, fructose, cellobiose, and maltose), or other fermentable carbohydrates. Sources include for example: corn, sugar cane, sugar beets, cassava, wheat, some crop residues and food waste. Not included within the scope of non-cellulosic biomass are the following if they contain less than 1 wt-% fermentable carbohydrate: distiller's dry grains, crop residues, plant matter and waste materials. These are typically considered cellulosic biomass materials. Cellulosic biomass materials are generated in large quantities as agricultural waste (e.g., from crops and grasses), wood, waste wood (e.g., from paper mills, logging residues, dead wood, forest brush clearing, orchards, and vineyards), and other wastes (e.g., municipal wastes).

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWING

The disclosure may be more completely understood in consideration of the following detailed description of various embodiments in connection with the accompanying drawings, in which:

FIG. 1 is a schematic flow diagram of one illustrative embodiment of the method according to the present disclosure;

FIG. 2 is a schematic flow diagram of a second illustrative embodiment of the method according to the present disclosure;

FIG. 3 is a schematic flow diagram of a third illustrative embodiment of the method according to the present disclosure;

FIG. 4 is a schematic flow diagram of a fourth illustrative embodiment of the method according to the present disclosure; and

FIG. 5 is a schematic perspective view of an illustrative membrane extraction module useful for practicing the methods disclosed herein.

### DETAILED DESCRIPTION OF ILLUSTRATIVE EMBODIMENTS

The methods according to the present disclosure are useful, for example, for recovering butanol (1-butanol or isobutanol), and preferably increasing the rate and/or yield of its production, from non-cellulosic biomass. Non-cellulosic biomass contains greater than 1 wt-% starch, dextrin, sugars (e.g., dextrose, sucrose, xylose, fructose, cellobiose, and maltose), or other fermentable carbohydrates. Sources include for example: corn, sugar cane, sugar beets, cassava, wheat, some crop residues and food waste. Not included within the scope of non-cellulosic biomass are the following if they contain less than 1 wt-% fermentable carbohydrate: distiller's dry grains, crop residues, plant matter and cellulosic waste materials. These are typically considered cellulosic biomass materials. Exemplary sources of cellulosic biomass include waste wood or bark, waste tree trunk chips from pulp or paper mills, forest waste (e.g., roots, branches, and foliage), orchard and vineyard trimmings, stalks and leaves (i.e., stover) from cotton plants, bamboo, rice, wheat, and corn, waste agricultural products (e.g., rice, wheat, and corn), agricultural byproducts (e.g., bagasse and hemp), and waste paper (e.g., newspaper, computer paper, and cardboard boxes). A common source of cellulosic biomass is corn stover. Some of these cellulosic materials (e.g., softwood and hardwood materials and crops) are lignocellulosic materials that contain lignin, cellulose, and hemicellulose.

1-Butanol and/or isobutanol can be produced via fermentation of an aqueous mixture containing carbohydrates derived from one or more sources of non-cellulosic biomass (e.g., corn-derived or sugar cane-derived sugars or possibly other starch-based sugars). An aqueous mixture that includes carbohydrates from non-cellulosic biomass may be obtained by known methods of digestion of non-cellulosic biomass. Such known digestion methods typically use enzymes such as amylase and glucoamylase at elevated temperatures.

The fermentation of digested non-cellulosic biomass can occur using a butanol-producing microorganism that is either native or engineered, e.g., Clostridium acetobutylicum, Clostridium beijerinckii, yeast, or E. Coli. Typically, either 1-butanol or isobutanol is produced depending on the specific microorganism. Unfortunately, butanol is a powerful feedback inhibitor of the microorganisms that produce it. For example, butanol concentration of as low as 2 wt-% can shut down fermentation. When butanol is continuously extracted from the fermentation broth using, for example, membrane solvent extraction as described herein, this feedback inhibition by butanol can be reduced, resulting in acceleration of the fermentation rate and/or enhanced butanol yield. After extraction, the butanol and a slight amount of water can be recovered, for example, by flash separation, vacuum distillation or other downstream enrichment processes. Significantly, this can result in lower overall energy of separation of the butanol compared to separation by conventional distillation.

A fermentation system suitable for use in the methods described herein can be any of a wide variety. This can include, for example, single-tank batch fermentation, multiple-tank batch fermentation, single-tank fed-batch fermentation, multiple-tank fed batch fermentation, single-tank continuous fermentation, or multiple-tank continuous fermentation.

Suitable microorganisms for use in a fermentation system that uses non-cellulosic biomass for the production of butanol include those described in the article by Chkwuemeka et al., “Bioproduction of Butanol from Biomass: from Genes to Bioreactors,” Current Opinion in Biotechnology, 2007, 18:220-227, and in the article by Lee et al. “Fermentive Butanol Production by Clostridia,” Biotechnology and Bioengineering, 2008, 101:209-228, as well as the following: Clostridia acetobutylicum, both native and engineered; Clostridia beijerinckii, both native and engineered; engineered E. Coli; engineered Bacillus subtilis; and engineered Saccharomyces cerevisiae (yeast). These are referred to herein as “butanol-fermenting microorganisms” or “butanol-producing microorganisms.” Preferred such microorganisms include native or engineered Clostridium acetobutylicum, Clostridium beijerinckii, yeast, or E. Coli. Mixtures of microorganisms can be used if it is desirable to produce a mixture of 1-butanol and isobutanol.

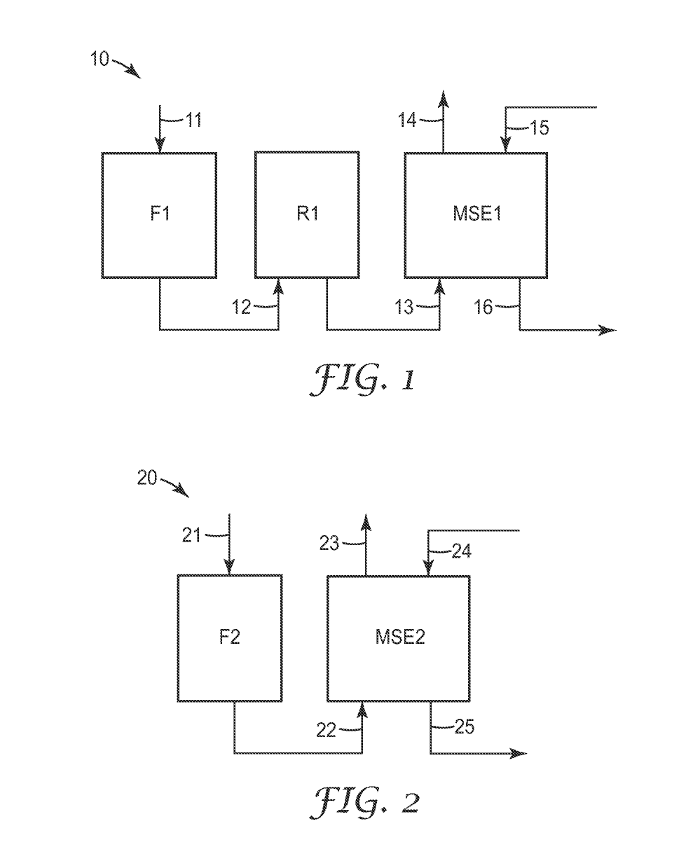

A process flow diagram 10 of an exemplary embodiment of the method according to the present disclosure is shown in FIG. 1. In flow diagram 10, an aqueous mixture, which contains carbohydrates, such as glucose, other sugars, and their oligomers, derived from non-cellulosic biomass (e.g., by enzymatic digestion), in water with other nutrients (such as ammonia, metallic ions, and vitamins), is introduced along line 11 into fermenter F1 along with one or more microorganisms for carrying out the fermentation. Fermentation broth containing butanol (first fermentation broth) is then transported to reservoir R1 along line 12 and collected therein. The fermentation broth will typically include 2 wt-% or less butanol. This fermentation broth is then directed along line 13 into membrane solvent extraction unit MSE1. In extractor MSE1, first fermentation broth (introduced along line 13) and a solvent extractant (introduced along line 15) are brought into intimate contact with each other such that the butanol produced partitions between the fermentation broth and the extractant. The resultant mixture of solvent extractant and the produced butanol (i.e., the extract) is then transported along line 16 for recovery, such as through flash separation, vacuum distillation, other downstream butanol enrichment process, or combinations thereof. In this process, after passing through MSE1, the fermentation broth with butanol extracted therefrom (i.e., the second fermentation broth has a lower concentration of butanol than the first fermentation broth) is removed along line 14 but is not recycled back into the fermenter. As a result, butanol enrichment is expected to occur relative to the final titer of butanol in the fermentation broth. For example, an increase from 2 wt-% up to 20 wt-%, or even as high as 50 wt-%, is possible.

A process flow diagram 20 of an exemplary embodiment of the method according to the present disclosure is shown in FIG. 2. In flow diagram 20, an aqueous mixture, which contains carbohydrates, such as glucose, other sugars, and their oligomers, derived from non-cellulosic biomass (e.g., by enzymatic digestion), in water with other nutrients (such as ammonia, metallic ions, and vitamins), is introduced along line 21 into fermenter F2 along with one or more microorganisms for carrying out the fermentation. First fermentation broth containing butanol (typically, 2 wt-% or less) is then transported directly to membrane solvent extraction unit MSE2 along line 22 (without the use of a storage reservoir as shown in FIG. 1). In extractor MSE2, fermentation broth (introduced along line 22) and a solvent extractant (introduced along line 24) are brought into intimate contact with each other such that the butanol produced partitions between the fermentation broth and the solvent extractant. The resultant mixture of extractant and butanol (i.e., the extract) is then transported along line 25 for recovery, such as through flash separation, vacuum distillation, other downstream butanol enrichment (i.e., concentration) processes, or combinations thereof. In this process, after passing through MSE2, the fermentation broth with butanol extracted therefrom (i.e., the second fermentation broth has a lower concentration of butanol than the first fermentation broth) is removed along line 23 but is not recycled. As a result, butanol enrichment is expected to occur, as with the process flow diagram 10 shown in FIG. 1.

A process flow diagram 30 of an exemplary embodiment of the method according to the present disclosure is shown in FIG. 3. In flow diagram 30, an aqueous mixture, which contains carbohydrates, such as glucose, other sugars, and their oligomers, derived from non-cellulosic biomass (e.g., by enzymatic digestion), in water with other nutrients (such as such as ammonia, metallic ions, and vitamins), is introduced along line 31 into fermenter F3 along with one or more microorganisms for carrying out the fermentation. First fermentation broth containing butanol is then transported to reservoir R3 along line 32 and collected therein. First fermentation broth will typically include 2 wt-% or less butanol. First fermentation broth is then directed along line 33 into membrane solvent extraction unit MSE3. In extractor MSE3, first fermentation broth (introduced along line 33) and a solvent extractant (introduced along line 35) are brought into intimate contact with each other such that the butanol produced partitions between the fermentation broth and the extractant. The resultant mixture of solvent extractant and the produced butanol (i.e., the extract) is then transported along line 36 for recovery, such as through flash separation, vacuum distillation, other downstream butanol enrichment process, or combinations thereof. In this process, after passing through MSE3, the second fermentation broth (that which has had butanol extracted therefrom and thus has a lower concentration of butanol than the first fermentation broth) is removed along line 34 and is recycled back into fermenter F3 (along line 34). As a result, butanol enrichment (relative to the final titer of butanol in the fermentation broth, which is typically 2 wt-% or less) and fermentation acceleration (relative to a method that does not recycle the fermentation broth (i.e., a non-recycling method)) are expected to occur using this process. For example, an increase from 2 wt-% up to 20 wt-% or even as high as 50 wt-% is possible, and at least a 2-fold increase in acceleration of butanol production is possible. An increase in the rate of delivery of carbohydrate starting materials (from the digested non-cellulosic biomass) in feed stream introduced along line 31 will typically be used to sustain this acceleration.

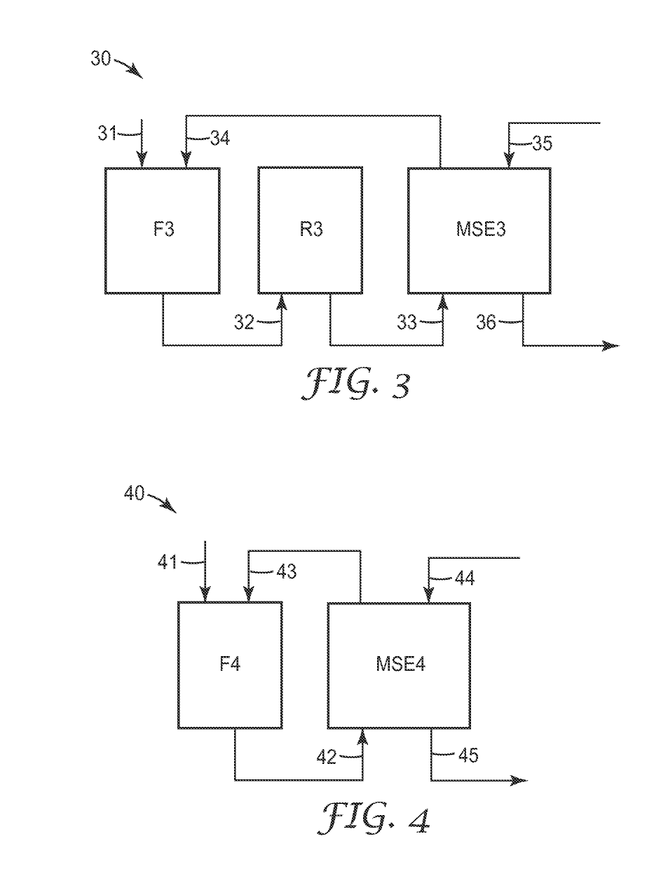

A process flow diagram 40 of an exemplary embodiment of the method according to the present disclosure is shown in FIG. 4. In flow diagram 40, an aqueous mixture, which contains carbohydrates, such as glucose, other sugars, and their oligomers, derived from non-cellulosic biomass (e.g., by enzymatic digestion), in water with other nutrients (such as ammonia, metallic ions, and vitamins), is introduced along line 41 into fermenter F4 along with one or more microorganisms for carrying out the fermentation. First fermentation broth containing butanol (typically 2 wt-% or less) is then transported directly into membrane solvent extraction unit MSE4 along line 42. In extractor MSE4, first fermentation broth (introduced along line 42) and a solvent extractant (introduced along line 44) are brought into intimate contact with each other such that the butanol produced partitions between the fermentation broth and the extractant. The resultant extract (mixture of solvent extractant and the produced butanol) is then transported along line 45 for recovery, such as through flash separation, vacuum distillation, other downstream butanol enrichment processes, or combinations thereof . In this process, after passing through MSE4, the second fermentation broth (having had butanol extracted therefrom and thus has a lower concentration of butanol than the first fermentation broth) is removed along line 43 and is recycled back into fermenter F4 (along line 43). As a result, butanol enrichment and fermentation acceleration are expected to occur using this process, as with the process flow diagram 30 shown in FIG. 3. As with the process flow diagram 30 in FIG. 3, an increase in the rate of delivery of carbohydrate starting materials (from the enzymatic digestion of non-cellulosic biomass) in feed stream introduced along line 41 will typically be used to sustain this acceleration.

The first extractant comprises a straight-chain or branched alcohol having from 7 to 12 (in some embodiments 8 to 12 or 8 to 11) carbon atoms. In preferred embodiments, the extractant has a boiling point that is at least 30° C. higher than the butanol produced (or the higher boiling butanol produced if a mixture is produced). In some of these embodiments, the straight-chain or branched alcohol is a primary alcohol. In some embodiments, the first extractant comprises a straight-chain alcohol having from 7 to 12 (in some embodiments 8 to 12 or 8 to 11) carbon atoms. In some of these embodiments, the first extractant comprises at least one of 2-octanol, 2-ethyl-1-hexanol, 1-nonanol, 2,6-dimethyl-4-heptanol, 1-decanol, 4-decanol, 2-propyl-1-heptanol, or combinations thereof. For isobutanol, preferred first extractants include 2-octanol, 2-ethyl-1-hexanol, 1-nonanol, 2,6-dimethyl-4-heptanol, 1-decanol, 4-decanol, and 2-propyl-1-heptanol. For 1-butanol, preferred first extractants include 2-octanol, 2-ethyl-1-hexanol, 2,6-dimethyl-4-heptanol, 4-decanol, and 2-propyl-1-heptanol. Various combinations of such alcohols could be used if desired.

In some embodiments of the methods disclosed herein, including the methods described above in connection with FIGS. 1 through 4, the methods can further include recovering at least a portion of the butanol (e.g., isobutanol and/or 1-butanol). As described above, the butanol can be concentrated by flash separation and/or vacuum distillation, for example.

In some embodiments, a portion of the first extractant can become entrained in the second fermentation broth. In such embodiments, a method of the present disclosure could further include, at least partially extracting the entrained first extractant from the second fermentation broth with a second extractant by a second liquid-liquid extraction, which can be through the use of a membrane extraction process. Exemplary second extractants include dodecane and decane, although dodecane is typically preferred.

A variety of porous membranes and membrane extraction apparatuses may be useful for practicing the present disclosure. In general, the rate of extraction depends on the area of the liquid-liquid interface. Thus, membrane extraction apparatus designs that have large membrane surface areas are typically desirable, although designs having relatively smaller membrane surface areas may also be useful.

The following embodiments of porous membranes and apparatuses may be useful for the first liquid-liquid extraction (that is, the extraction of butanol) or the optional second liquid-liquid extraction (that is, the extraction of the entrained first extractant). The membrane extraction apparatus may be of any design as long as the extractant and aqueous solution to be extracted have a liquid-liquid interface within at least one pore, typically a plurality of pores, of the porous membrane.

To facilitate formation of an interface between the aqueous solution and the extractant within the porous membrane, whichever of the aqueous solution or the extractant wets the membrane least well may be maintained at higher pressure than the other. For example, in the case of a hydrophobic porous membrane the aqueous solution may have a higher fluid pressure than the extractant. This pressure differential should typically be sufficient to substantially immobilize the interface between the aqueous solution and extractant, but preferably not large enough to cause damage to the porous membrane. The pressure differential may be achieved by a variety of known methods including a restriction valve (e.g., a back-pressure valve on an extract outlet port), a fluid height differential, or the like. If present, the pressure differential between the aqueous solution and the extractant may be, for example, at least 10 cm water at 4° C. (1 kPa), at least 1 pound per square inch (psi) (6.9 kPa), and may be up to 11 psi (76 kPa), although higher and lower pressures may also be used.

Microporous membranes useful for practicing the present disclosure typically have micrometer-sized pores (that is, micropores) that extend between major surfaces of the membrane. The micropores may be, for example, isolated or interconnected. The microporous membrane may be formed from any material having micropores therethrough, for example, a microporous thermoplastic polymer. The microporous membrane may, for example, be flexible or rigid. In some embodiments according to the present disclosure, useful thermoplastic microporous membranes may comprise a blend of similar or dissimilar thermoplastic polymers, each optionally having a different molecular weight distribution (e.g., a blend of ultrahigh molecular weight polyethylene and high molecular weight polyethylene).

Micropore size, thickness, and composition of the microporous membranes typically determine the rate of extraction in the methods disclosed herein. The size of the micropores of the microporous membrane should be sufficiently large to permit contact between the aqueous solution and the extractant within the micropores (e.g., to form a liquid-liquid extraction interface), but not so large that flooding of the aqueous solution occurs through the microporous membrane into the extractant occurs. Microporous membranes useful for practice of the present invention may be, for example, hydrophilic or hydrophobic. Microporous membranes can be prepared by methods well known in the art and are described in, for example, U.S. Pat. No. 3,801,404 (Druin et al.), U.S. Pat. No. 3,839,516 (Williams et al.), U.S. Pat. No. 3,843,761 (Bierenbaum et al.), U.S. Pat. No. 4,255,376 (Soehngen et al.), U.S. Pat. No. 4,257,997 (Soehngen et al.), U.S. Pat. No. 4,276,179 (Soehngen), U.S. Pat. No. 4,973,434 (Sirkar et al.), and/or are widely commercially available from suppliers such as, for example, Celgard, Inc. (Charlotte, N.C.); Tetratec, Inc. (Ivyland, Pa.); Nadir Filtration GmbH (Wiesbaden, Germany); or Membrana, GmbH (Wuppertal, Germany). Exemplary hydrophilic membranes include membranes of microporous polyamide (e.g., microporous nylon), microporous polycarbonate, microporous ethylene vinyl alcohol copolymer, and microporous hydrophilic polypropylene. Exemplary hydrophobic membranes include membranes of microporous polyethylene, microporous polypropylene (e.g., thermally induced phase separation microporous polypropylene), and microporous polytetrafluoroethylene.

Typically, the mean pore size of useful microporous membranes (e.g., as measured according to ASTM E1294-89 (1999) “Standard Test Method for Pore Size Characteristics of Membrane Filters Using Automated Liquid Porosimeter”) may be greater than about 0.07 micrometer (e.g., greater than 0.1 micrometer or greater than 0.25 micrometer), and may be less than 1.4 micrometers (e.g., less than 1.0 micrometer, less than 0.4 micrometer or less than 0.3 micrometer), although microporous membranes having larger or smaller mean pore sizes may also be used. In order to reduce emulsion formation and/or flooding across the membrane, the microporous membrane may be substantially free of pores, tears, or other holes that exceed 100 micrometers in diameter.

Useful microporous membranes typically have a porosity in a range of from at least about 20 percent (e.g., at least 30 percent or at least 40 percent) up to 80 percent, 87 percent, or even 95 percent, based on the volume of the microporous membrane. Typically, useful microporous membranes have a thickness of at least about 25 micrometers (e.g., at least 35 micrometers or at least 40 micrometers), and/or may have a thickness of less than about 80 micrometers (e.g., less than 60 micrometers or even less than 50 micrometers), although membranes of any thickness may be used. Typically, microporous membranes should be mechanically strong enough, alone or in combination with an optional porous support member, to withstand any pressure difference that may be imposed across the microporous membrane under the intended operating conditions.

Multiple microporous membranes may be used in series or in parallel for any of the liquid-liquid extractions disclosed herein. Exemplary membrane forms include sheets, bags, and tubes and may be substantially planar or nonplanar (e.g., pleated, spiral wound cartridge, plate-frame, or hollow fiber bundle). In some embodiments of methods disclosed herein, a microporous membrane may comprise a microporous hollow fiber membrane as described in, for example, U.S. Pat. No. 4,055,696 (Kamada et al.), U.S. Pat. No. 4,405,688 (Lowery et al.), and U.S. Pat. No. 5,449,457 (Prasad). Of course, the nature of the extraction apparatus (e.g., shape, size, components) may vary depending on the form of the membrane chosen.

The microporous membrane may comprise at least one hydrophobic (that is, not spontaneously wet out by water) material. Exemplary hydrophobic materials include polyolefins (e.g., polypropylene, polyethylene, polybutylene, copolymers of any of the forgoing and, optionally, an ethylenically unsaturated monomer), and combinations thereof. If the microporous membrane is hydrophobic, a positive pressure may be applied to the aqueous solution relative to the extractant to aid in wetting the microporous membrane.

In some embodiments of the methods disclosed herein, the microporous membrane may be hydrophilic, for example, a hydrophilic microporous polypropylene membrane having a nominal average pore size in a range of from 0.2 to 0.45 micrometers (e.g., as marketed under the trade designation “GH POLYPRO MEMBRANE” by Pall Life Sciences, Inc., Ann Arbor, Mich.). If the microporous membrane is hydrophilic, positive pressure may be applied to the extractant relative to the aqueous solution to facilitate immobilization of the liquid-liquid interface within the membrane. Exemplary membranes include microporous membranes as described in U.S. Pat. No. 3,801,404 (Druin et al.), U.S. Pat. No. 3,839,516 (Williams et al.), U.S. Pat. No. 3,843,761 (Bierenbaum et al.), U.S. Pat. No. 4,255,376 (Soehngen), U.S. Pat. No. 4,257,997 (Soehngen et al.), and U.S. Pat. No. 4,276,179 (Soehngen), U.S. Pat. No. 4,726,989 (Mrozinski), U.S. Pat. No. 5,120,594 (Mrozinski), and U.S. Pat. No. 5,238,623 (Mrozinski).

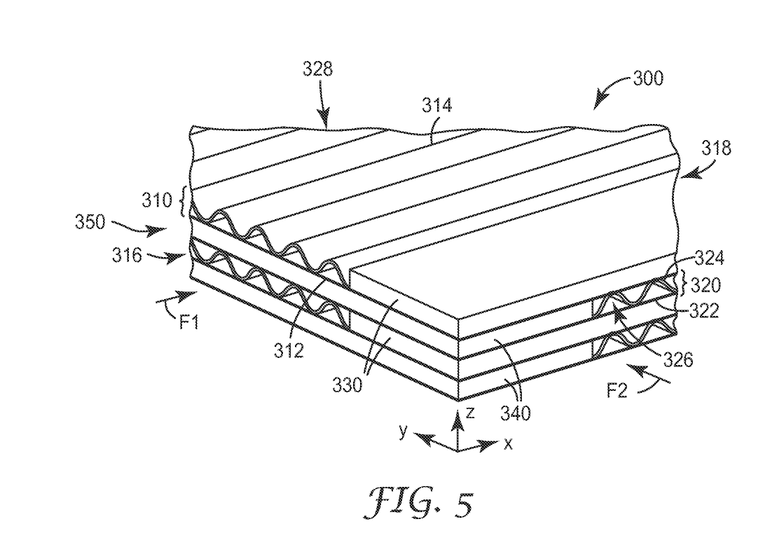

Suitable Membrane Solvent Extraction (MSE) Units suitable for use in the methods described herein include, for example, single MSE module or multiple MSE modules. Several useful microporous membrane extraction apparatuses are described, for example, in U.S. Pat. No. 7,105,089 (Fanselow et al.), and U.S. Pat. App. Pub. No. 2007/0119771 (Shukar et al.). An exemplary embodiment of a membrane extraction element (i.e., membrane solvent extraction unit) of a membrane extraction apparatus useful for practicing the present disclosure is shown in FIG. 5. The membrane extraction element 300 includes a first layer pair 310 and a second layer pair 320. The second layer pair 320 is disposed adjacent the first layer pair 310 forming a stack of layers 350. The stack of layers 350 has an x-, y-, and z-axis as shown in FIG. 5. The z-axis is the thickness direction of the stack of layers 350. The x-axis and y-axis are both in-plane axes of the stack of layers 350 and are orthogonal to one another in the illustrated embodiment.

The first layer pair 310 includes first polymeric microporous membrane 312 and a first flow channel layer 314 oriented in a first flow F₁ direction (along the x-axis of FIG. 5) having a fluid inlet 316 and a fluid outlet 318 disposed on first opposing sides of the extraction element 300 (along the y-axis of FIG. 5). Thus, in the illustrative embodiment shown in FIG. 5, the first flow F₁ direction is orthogonal to the first opposing sides of the liquid-liquid extraction element 300.

The second layer pair 320 includes a second polymeric microporous membrane 322 and a second flow channel layer 324 oriented in a second flow direction F₂ (along the y-axis of FIG. 5) different than the first flow direction F₁ and having a fluid inlet 326 and a fluid outlet 328 disposed on second opposing sides (along the x-axis of FIG. 5) of the membrane extraction element 300. Thus, in the illustrative embodiment shown in FIG. 5, the second flow F₂ direction is orthogonal to the second opposing sides of the membrane extraction element 300. The first microporous membrane 312 is shown disposed between the first flow channel layer 314 and the second flow channel layer 324. In the illustrated embodiment, the first flow direction F₁ is orthogonal to the second flow direction F₂, but this is not required.

In many embodiments, the liquid-liquid extraction element 300 includes a plurality (two or more) of alternating first layer pairs 310 and second layer pairs 320. In some embodiments, the membrane extraction element 300 includes from 10 to 2000, or 25 to 1000, or 50 to 500 alternating first layer pairs 310 and second layer pairs 320 stacked in vertical registration (along the z-axis) where the first flow direction F₁ (along the x-axis) is orthogonal to the second flow direction F₂ (along the y-axis).

The flow channel layers 314, 324 and the microporous membrane layers 312, 322 have layer thicknesses (along the z-axis) of any useful value. In many embodiments, the first flow channel layer 314 and the second flow channel layer 324 each has a thickness in a range from 10 to 250, or 25 to 150 micrometers. In many embodiments, the first polymeric microporous membrane 312 and the second polymeric microporous membrane 322 each has a thickness in a range from 1 to 200, or 10 to 100 micrometers. The extraction element 300 has an overall thickness (along the z-axis) of any useful value. In some embodiments, the extraction element 300 has an overall thickness (along the z-axis) in a range from 5 to 100, or 10 to 50 centimeters.

The membrane extraction element 300 can have any useful shape (e.g., a rectilinear shape). The extraction element 300 has a width (along the y-axis) and a length (along the x-axis) of any useful value. In some embodiments, the extraction element 300 has an overall width (along the y-axis) in a range from 10 to 300, or 50 to 250 centimeters. In some embodiments, the extraction element 300 has an overall length (along the x-axis) in a range from 10 to 300, or 50 to 250 centimeters. In one embodiment, the extraction element 300 length is equal or substantially equal to its width.

The first and second flow channel layers 314, 324 can be formed of the same or different material and take the same or different forms, as desired. The first and second flow channel layers 314, 324 can allow liquid to flow between first and second microporous membranes 312, 322. In many embodiments, the first and second flow channel layers 314, 324 can be structured such that the first and second flow channel layers 314, 324 form flow channels between the microporous membranes 312, 322. In some embodiments, the first and second flow channel layers 314, 324 are non-porous and formed of a polymeric material (e.g., a polyolefin).

In some embodiments, the first and second flow channel layers 314, 324 are corrugated (having parallel alternating peaks and valleys) to provide flow channels between the microporous membranes 312, 322. In many embodiments, the corrugations provide flow channels that are parallel the flow direction. These corrugations can have any useful pitch (distance between adjacent peaks or valleys). In some embodiments, the corrugations have a pitch in a range from 0.05 to 1, or from 0.1 to 0.7 centimeter. The corrugations can be formed by any useful method (e.g., embossing or molding).

As shown in FIG. 5, an exemplary configuration of the extraction element 300 includes a first layer pair 310 having first planar polymeric microporous membrane 312 and a first corrugated flow channel layer 314 oriented in a first flow F₁ direction (along the x-axis of FIG. 5). Thus, in the illustrative embodiment shown in FIG. 5, the first flow F₁ direction is parallel to the corrugations of the first corrugated flow channel layer 314. The second layer pair 320 includes a second planar polymeric microporous membrane 322 and a second corrugated flow channel layer 324 oriented in a second flow direction F₂ (along the y-axis of FIG. 5) orthogonal to the first flow direction F₁ and parallel to the corrugations of the second corrugated flow channel layer 324. Thus, in the illustrative embodiment shown, the first flow direction F₁ is orthogonal to the second flow direction F₂, and the corrugations of the first corrugated flow channel layer 314 are orthogonal to the corrugations of the second corrugated flow channel layer 324.

The extraction element 300 can optionally include layer seals 330, 340 disposed along the selected edges of the extraction element 300. First layer seals 330 can be formed between the porous membrane of one layer, and the flow channel layer below it (in the flow direction of that flow channel layer) along opposing sides of the liquid-liquid extraction element 300. Second layer seals 340 can be formed between the porous membrane of one layer, and the flow channel layer below it (in the flow direction of that flow channel layer) along opposing sides of the extraction element 300. In some embodiments, first and second layer seals, 330, 340 alternate on opposing sides, as shown in FIG. 5.

In some embodiments, layer seals 330, 340 between the layers can be beads of adhesive, a sonic seal, or a heat seal. Thus, a two-directional liquid-liquid extraction flow module can be created, in which a first fluid flows through the module in a first direction, passing through the corrugated spacers and porous membrane of every other layer, contacting the porous membrane layers uniformly on one side; and a second fluid is directed to flow through the liquid-liquid extraction module in a second direction (often orthogonal) to the first direction, passing through the corrugated spacers of layers alternate to the first, contacting the membrane layers uniformly on the other side.

In some embodiments, a first porous non-woven layer (not shown) is disposed between the first polymeric microporous membrane 312 and the first flow channel layer 314 and a second porous non-woven layer (not shown) is disposed between the second polymeric microporous membrane 322 and the second flow channel layer 324. This porous non-woven layer can assist in reinforcing the microporous membrane layer and/or the flow channel layer. The porous non-woven layer can be any useful material such as, for example, a spunbond layer. This porous non-woven layer can be optionally attached (adhesive, ultrasonic seal, heat seal, and the like) to the polymeric microporous membrane and/or flow channel layer.

In some embodiments, a first vessel (not shown) containing a volume of a fermentation broth is in fluid communication with a plurality of first layer pairs 310. In some of these embodiments, a second vessel (not shown) containing a volume of a first extractant is in fluid communication with a plurality of second layer pairs 320. The first vessel may be connected to a first entrance manifold (not shown) in fluid communication with the first fluid inlet 316 of each first layer pair 310. The fermentation broth may enter all of the first layer pairs 310 through the manifold. In some embodiments of a membrane extraction system disclosed herein, a first exit manifold, through which a second fermentation broth exits from all of the first layer pairs 310, is in fluid communication with the first fluid outlet 318 of each first layer pair 310. In some embodiments of a membrane extraction system disclosed herein, a second entrance manifold (not shown) in fluid communication with the second fluid inlet 326 of each second layer pair 320 is connected to the second vessel and allows the first extractant to enter all of the second layer pairs 320. In some embodiments, a second exit manifold (not shown), through which an extract exits from all of the second layer pairs 320, is in fluid communication with the second fluid outlet 328 of each second layer pair 320.

The methods according to the present disclosure preferably increase the rate of fermentation to produce butanol (e.g., isobutanol and/or 1-butanol) and/or increase the yield of butanol produced in a fermentation process. That is, when methods according to the present disclosure are used to at least partially remove butanol from the fermenter, production of butanol can occur at a higher rate than when fermentation is carried out on aqueous mixtures from non-cellulosic biomass not subjected to extraction of butanol from the fermenter.

### Selected Embodiments of the Disclosure

Embodiment 1 is a method of producing butanol, the method comprising:

introducing an aqueous mixture comprising carbohydrates obtained from non-cellulosic biomass into a fermenter;

fermenting the aqueous mixture to provide a first fermentation broth, the fermentation broth comprising:
a microorganism for producing butanol;
carbohydrates from the non-cellulosic biomass; and
butanol; and

at least partially extracting the butanol from the first fermentation broth with a first solvent extractant by a first liquid-liquid extraction through a first porous membrane to provide a first extract and a second fermentation broth;
wherein the first solvent extractant comprises a straight-chain or branched alcohol having from 7 to 12 carbon atoms;
wherein the second fermentation broth has a lower concentration of the butanol than the first fermentation broth;
wherein the first liquid-liquid extraction is carried out in a liquid-liquid extraction element comprising:
   a plurality of first layer pairs, each first layer pair comprising:
      a first polymeric microporous membrane; and
      a first flow channel layer oriented in a first flow direction having a first fluid inlet and a first fluid outlet disposed on first opposing sides of the liquid-liquid extraction element; and
   a plurality of second layer pairs, with at least one second layer pair being disposed between two first layer pairs and at least one first layer pair being disposed between two second layer pairs so as to form a stack of layers, each second layer pair comprising:
      a second polymeric microporous membrane; and
      a second flow channel layer oriented in a second flow direction different than the first flow direction and having a second fluid inlet and a second fluid outlet disposed on second opposing sides of the extraction element.

Embodiment 2 is the method of embodiment 1 wherein the butanol produced is 1-butanol.

Embodiment 3 is the method of embodiment 1 or 2 further comprising directing the second fermentation broth back into the fermenter thereby increasing the rate of butanol production relative to a non-recycling method.

Embodiment 4 is the method of any of the preceding embodiments wherein the first extractant has a boiling point that is at least 30° C. higher than the butanol produced, or, if a mixture is produced, 30° C. higher than the higher boiling butanol produced.

Embodiment 5 is the method of any of the preceding embodiments wherein the first extractant comprises a straight-chain or branched alcohol having from 8 to 11 carbon atoms.

Embodiment 6 is the method of embodiment 5 wherein the first extractant comprises a 2-octanol, 2-ethyl-1-hexanol, 1-nonanol, 2,6-dimethyl-4-heptanol, 1-decanol, 4-decanol, 2-propyl-1-heptanol, or combinations thereof.

Embodiment 7 is the method of any of the preceding embodiments wherein the microorganism for producing butanol comprises native or engineered Clostridium acetobutylicum, Clostridium beijerinckii, yeast, E. Coli, or a combination thereof.

Embodiment 8 is the method of any of the preceding embodiments wherein a portion of the first extractant becomes entrained in the second fermentation broth, the method further comprises at least partially extracting the entrained first extractant from the second fermentation broth with a second extractant by a second liquid-liquid extraction.

Embodiment 9 is the method of embodiment 8 wherein the second extractant comprises dodecane.

Embodiment 10 is the method of any of the preceding embodiments wherein the non-cellulosic biomass comprises corn, sugar cane, sugar beets, cassava, wheat, or mixtures thereof.

Embodiment 11 is a method of recovering butanol from a fermentation broth, the method comprising:
introducing an aqueous mixture comprising carbohydrates obtained from non-cellulosic biomass into a fermenter;
fermenting the aqueous mixture to provide a first fermentation broth, the fermentation broth comprising:
   a microorganism for producing butanol;
   carbohydrates from the non-cellulosic biomass; and
   butanol;
at least partially extracting the butanol from the first fermentation broth with a first solvent extractant by a first liquid-liquid extraction through a first porous membrane to provide a first extract and a second fermentation broth; and
recovering at least a portion of the butanol from the first extract;
wherein the first solvent extractant comprises a straight-chain or branched alcohol having from 7 to 12 carbon atoms;
wherein the second fermentation broth has a lower concentration of the butanol than the first fermentation broth;
wherein the first liquid-liquid extraction is carried out in a liquid-liquid extraction element comprising:
   a plurality of first layer pairs, each first layer pair comprising:
      a first polymeric microporous membrane; and
      a first flow channel layer oriented in a first flow direction having a first fluid inlet and a first fluid outlet disposed on first opposing sides of the liquid-liquid extraction element; and
   a plurality of second layer pairs, with at least one second layer pair being disposed between two first layer pairs and at least one first layer pair being disposed between two second layer pairs so as to form a stack of layers, each second layer pair comprising:
      a second polymeric microporous membrane; and
      a second flow channel layer oriented in a second flow direction different than the first flow direction and having a second fluid inlet and a second fluid outlet disposed on second opposing sides of the extraction element.

Embodiment 12 it the method of embodiment 11 wherein recovering the butanol comprises concentrating it by flash separation and/or vacuum distillation.

Embodiment 13 is the method of embodiment 11 or 12 wherein the first extractant has a boiling point that is at least 30° C. higher than the butanol produced, or, if a mixture is produced, 30° C. higher than the higher boiling butanol produced.

Embodiment 14 is the method of any of the preceding embodiments 11 through 13 wherein the first extractant comprises a straight-chain or branched alcohol having from 8 to 11 carbon atoms.

Embodiment 15 is the method of embodiment 14 wherein the first extractant comprises a 2-octanol, 2-ethyl-1-hexanol, 1-nonanol, 2,6-dimethyl-4-heptanol, 1-decanol, 4-decanol, 2-propyl-1-heptanol, or combinations thereof.

Embodiment 16 is the method of any of the preceding embodiments 11 through 15 wherein the microorganism for producing butanol comprises native or engineered Clostridium acetobutylicum, Clostridium beijerinckii, yeast, E. Coli, or a combination thereof.

Embodiment 17 is the method of any of the preceding embodiments 11 through 16 wherein a portion of the first extractant becomes entrained in the second fermentation broth, the method further comprises at least partially extracting the entrained first extractant from the second fermentation broth with a second extractant by a second liquid-liquid extraction.

Embodiment 18 is the method of embodiment 17 wherein the second extractant comprises dodecane.

Embodiment 19 is the method of any of the preceding embodiments 11 through 18 wherein the non-cellulosic biomass comprises corn, sugar cane, sugar beets, cassava, wheat, or mixtures thereof.

Embodiment 20 is the method of any of the preceding embodiments 11 through 19 wherein the butanol produced is isobutanol.

Objects and advantages of this disclosure are further illustrated by the following non-limiting examples, but the particular materials and amounts thereof recited in these examples, as well as other conditions and details should not be construed to unduly limit this disclosure.

### EXAMPLES

Unless otherwise noted, all parts, percentages, ratios, etc. in the Examples and the rest of the specification are by weight. These abbreviations are used in the following examples: g=grams, min=minutes, hr=hour, mL=milliliter, L=liter. If not otherwise indicated in the table, below, chemicals were obtained from Sigma-Aldrich, St. Louis, Mo.

### Example 1-8

### Solvents with High Distribution Coefficient and High Selectivity for 1-Butanol

### Method:

Oleyl alcohol, 2-ethyl-1-hexanol and 4-decanol were obtained from Alfa Aesar (Ward Hill, Mass.). Mesitylene, decane, 2-octanol, 1-nonanol, 2,6-dimethly-4-heptanol and 1-decanol were obtained from Sigma Aldrich (St. Louis, Mo.). 2-propyl-1-heptanol was obtained from BASF (Florham Park, N.J.). Samples of 2 mL of 2 wt % 1-butanol solution in water and 2 mL of each solvent were added to a 6 mL glass vial and shaken thoroughly. After shaking, samples were incubated at room temperature (25° C.) overnight. Samples from each phase were analyzed by a gas chromatograph (HP 6890 system, Agilent Technologies, Santa Clara, Calif.) equipped with a thermal conductivity detector and a wax column (DB-WAX, Agilent Technologies) to quantify 1-butanol and water concentrations in both phases.

### Distribution Coefficient:

The distribution coefficient for 1-butanol, K_{DB}, is defined as:

K_{DB}=[BuOH]_{SLV}/[BuOH]_{AQU}

where [BuOH]_{SLV} is the weight percent of 1-butanol in the solvent phase and [BuOH]_{AQU} is the weight percent of 1-butanol in the aqueous phase.

In the same fashion, the distribution coefficient for water, K_{DW}, is defined as:

K_{DW}=[H₂O]_{SLV}/[H₂O]_{AQU}

where [H₂O]_{SLV} is the weight percent of water in the solvent phase and [H₂O]_{AQU} is the weight percent of water in the aqueous phase.

### Separation Factor (Alpha):

Separation factor, α or alpha, is defined as the ratio of the 1-butanol distribution coefficient to that of water:

$$α = \frac{{K}_{DB}}{{K}_{DW}}$$

TABLE 1
Experimental results: distribution coefficient and selectivity of 1-butanol at 25° C.

| | | [ButOH]_{Aq} | [H₂O]_{Aq} | [ButOH]_{Org} | [H₂O]_{Org} | | | |
|---|---|---|---|---|---|---|---|---|
| Example | Solvent | (wt %) | (wt %) | (wt %) | (wt %) | KDB | KDW | Alpha |
| Comparative 1 | Oleyl | 0.57% | 99.43% | 1.72% | 0.96% | 2.99 | 0.010 | 308 |
| | alcohol | | | | | | | |
| Comparative 2 | Mesitylene | 1.25% | 98.75% | 0.84% | n.d. | 0.67 | −0.002 | n.d. |
| Example 1 | Decane | 1.69% | 98.31% | 0.33% | n.d. | 0.20 | −0.005 | n.d. |
| Example 2 | 2-Octanol | 0.21% | 99.79% | 2.12% | 3.31% | 9.94 | 0.033 | 299 |
| Example 3 | 2-Ethyl-1- | 0.24% | 99.76% | 2.07% | 1.92% | 8.59 | 0.019 | 447 |
| | hexanol | | | | | | | |
| Example 4 | 1-Nonanol | 0.27% | 99.73% | 1.98% | 3.58% | 7.37 | 0.036 | 205 |
| Example 5 | 2,6- | 0.28% | 99.72% | 2.07% | 1.02% | 7.42 | 0.010 | 724 |
| | Dimethyl- | | | | | | | |
| | 4-heptanol | | | | | | | |
| Example 6 | 1-Decanol | 0.28% | 99.72% | 1.95% | 3.22% | 6.89 | 0.032 | 213 |
| Example 7 | 4-Decanol | 0.31% | 99.69% | 1.98% | 0.90% | 6.36 | 0.009 | 709 |
| Example 8 | 2-Propyl-1- | 0.30% | 99.70% | 1.88% | 1.11% | 6.19 | 0.011 | 557 |
| | heptanol | | | | | | | |

Note:
“n.d.” means “not determined” due to the significantly low concentrations of water in the solvent phase

Oleyl alcohol has been the benchmark solvent for butanol extraction. Mesitylene was referenced in Kraemer et al., “Separation of Butanol from Acetone-Butanol-Ethanol Fermentation by a Hybrid Extraction-Distillation Process,” Computer Aided Chemical Engineering, (2010) 28:7-12. Mesitylene and decane had water concentrations in the solvent phase below the detection limit, thus no alpha value was calculated. The experimental results in Table 1 indicate the following 5 solvents had equivalent or higher performance on both K_{CB} and selectivity than oleyl alcohol (Comparative 1): 2-octanol, 2-ethyl-1-hexanol, 2,6-dimethyl-4-heptanol, 4-decanol, and 2-propyl-1-heptanol.

### Example 9-16

### Solvents with High Distribution Coefficient and High Selectivity for Isobutanol

### Method:

The same solvents were examined as extraction solvents for isobutanol. The same method described in Example 1-8 was used. The distribution coefficient for isobutanol, K_{DI}, is defined the same way as K_{DB}.

TABLE 2
Experimental results: distribution coefficient and selectivity of isobutanol at 25° C.

| | | [IsoButOH]_{Aq} | [H₂O]_{Aq} | [IsoButOH]_{Org} | [H₂O]_{Org} | | | |
|---|---|---|---|---|---|---|---|---|
| Example | Solvent | (wt %) | (wt %) | (wt %) | (wt %) | KDI | KDW | Alpha |
| Comparative 3 | Oleyl | 0.61% | 99.39% | 1.66% | 1.23% | 2.74 | 0.012 | 221 |
| | alcohol | | | | | | | |
| Comparative 4 | Mesitylene | 1.21% | 98.79% | 0.79% | 0.27% | 0.66 | 0.003 | 241 |
| Example 9 | Decane | 1.66% | 98.34% | 0.34% | 0.09% | 0.21 | 0.001 | 234 |
| Example 10 | 2-Octanol | 0.22% | 99.78% | 2.05% | 3.56% | 9.16 | 0.036 | 257 |
| Example 11 | 2-Ethyl-1- | 0.24% | 99.76% | 2.02% | 2.37% | 8.54 | 0.024 | 359 |
| | hexanol | | | | | | | |
| Example 12 | 1-Nonanol | 0.25% | 99.75% | 1.86% | 3.68% | 7.42 | 0.037 | 201 |
| Example 13 | 2,6- | 0.28% | 99.72% | 2.06% | 1.41% | 7.24 | 0.014 | 510 |
| | Dimethyl- | | | | | | | |
| | 4-heptanol | | | | | | | |
| Example 14 | 1-Decanol | 0.28% | 99.72% | 1.86% | 3.39% | 6.59 | 0.034 | 194 |
| Example 15 | 4-Decanol | 0.31% | 99.69% | 1.90% | 1.26% | 6.10 | 0.013 | 483 |
| Example 16 | 2-Propyl- | 0.29% | 99.71% | 1.87% | 1.39% | 6.38 | 0.014 | 456 |
| | 1-heptanol | | | | | | | |

The experimental results in Table 2 indicate the following 7 solvents showed equivalent or higher performance in both K_{DI} and selectivity than oleyl alcohol (Comparative 3): 2-octanol, 2-ethyl-1-hexanol, 1-nonanol, 2,6-dimethyl-4-heptanol, 1-decanol, 4-decanol, and 2-propyl-1-heptanol.

### Example 17

### Membrane Solvent Extraction of 1-Butanol

### Method:

1-Butanol was extracted using an 8-inch×8-inch×2-inch cross-flow membrane solvent extraction (MSE) module in a module housing unit (described in U.S. Pat. Pub. No. US 2007/0119771) with 2,6-dimethyl-4-heptanol as the extraction solvent. The MSE module had 1.007 m² of membrane surface area. Polypropylene porous membrane (average pore size of 0.35 micrometer, average porosity of 36.6%, thickness of 75 micrometer) made by the thermally-induced phase separation process (described in U.S. Pat. Nos. 4,726,989 and 5,120,594) was incorporated into the MSE module. A sample of 2,000 g of solution of 14 g/L 1-butanol in water was in an aqueous reservoir, which was connected via—pipes to and from the MSE module to form a circulating aqueous loop. 2,000 g of 2,6-dimethyl-4-heptanol was added to a solvent reservoir, which was connected via-pipes to the separate parts of the MSE module to form a circulating solvent loop. Solutions in the aqueous and solvent reservoirs were pumped by gear pumps at 250 mL/min and 1300 mL/min, respectively. Transmembrane pressure was controlled to be about 0.2 psi (higher pressure in aqueous phase). Solution temperatures were set at 50° C. During the MSE run, aqueous phase and solvent phase contacted one another within the porous membrane and solvent extraction of the butanol from aqueous phase to solvent phase occurred. Samples from the aqueous loop and the solvent loop were collected via sampling ports every 10 minutes. A gas chromatograph (HP 5890A, Agilent Technologies) equipped with a thermal conductivity detector and a wax column (19091-N-213, Agilent Technologies) was used to quantify 1-butanol and water concentrations in both phases.

### Results:

During membrane solvent extraction, 1-butanol was continuously extracted from the aqueous phase to the solvent phase (2,6-dimethyl-4-heptanol), as shown in Table 3. The concentration of 1-butanol in the solvent, [BuOH]_{SLV}, increased from 0 g/L at 0 min to 11.7 g/L at 90 min, while concentration of water in the solvent, [H₂O]_{SLV}, increased from 2.9 g/L at 10 min to 7.2 g/L at 90 min. During the MSE operation, no emulsion formation was observed. From these results, the expected 1-butanol concentration by flash separation of the solvent phase is calculated as [BuOH]_{SLV}/([BuOH]_{SLV}+[H₂O]_{SLV}) (Table 3). The expected butanol concentrations would be in the range of 56-66%, indicating significant 1-butanol enrichment from an initial 1.4 wt-% (13.9 g/L).

TABLE 3
Membrane solvent extraction of 1-butanol using
2.6-dimethyl-4-heptanol

| Time | [BuOH]_{Aq} | [BuOH]_{SLV} | [H₂O]_{SLV} | [BuOH]_{SLV}/ |
|---|---|---|---|---|
| (min) | (g/L) | (g/L) | (g/L) | ([BuOH]_{SLV} + [H₂O]_{SLV}) |
| 0 | 13.9 | 0.0 | n.d. | n.d. |
| 10 | 10.7 | 3.7 | 2.9 | 56.4% |
| 20 | 9.1 | 6.2 | 4.5 | 58.0% |
| 30 | 7.7 | 7.4 | 5.3 | 58.2% |
| 40 | 6.5 | 8.4 | 5.7 | 59.7% |
| 50 | 5.2 | 9.6 | 5.7 | 63.0% |
| 60 | 4.6 | 10.1 | 5.5 | 64.9% |
| 70 | 4.1 | 11.0 | 5.5 | 66.5% |
| 80 | 3.7 | 11.0 | 6.6 | 62.6% |
| 90 | 3.3 | 11.7 | 7.2 | 61.9% |

### Example 18

### Membrane Solvent Extraction of Isobutanol

### Method:

Isobutanol was extracted using a multi-layer cross-flow MSE unit with 2,6-dimethyl-4-heptanol. The method was the same as Example 17 except for the use of 14 g/L isobutanol in water, instead of 14 g/L 1-butanol in water.

### Results:

Throughout the MSE operation, isobutanol was continuously extracted from the aqueous phase to the solvent phase (2,6-dimethyl-4-heptanol), as shown in Table 4. In the isobutanol run, extraction actually happened before time 0 min as time 0 was defined as when the MSE established a stable condition of flow and pressure. Concentration of isobutanol in the solvent, [Iso-BuOH]_{SLV}, increased from 2.37 g/L at 0 min to 8.63 g/L at 90 min, while concentration of water in the solvent, [H₂O]_{SLV}, increased from 3.34 g/L at 0 min to 6.05 g/L at 90 min. During the MSE operation, no emulsion formation was observed. From these results, the expected iso-butanol concentration by flash separation of solvent phase is calculated by [Iso-BuOH]_{SLV}/([Iso-BuOH]_{SLV}+[H₂O]_{SLV}), (Table 4). The expected concentrations would be in the range of 46-58%, indicating significant isobutanol enrichment from an initial 1.0 wt-% (10.2 g/L).

TABLE 4
Membrane solvent extraction of isobutanol using
2.6-dimethyl-4-heptanol

| | | [Iso- | | [IsoBuOH]_{SLV}/ |
|---|---|---|---|---|
| Time | [Iso-BuOH]_{Aq} | BuOH]_{SLV} | [H₂O]_{SLV} | ([IsoBuOH]_{SLV} + |
| (min) | (g/L) | (g/L) | (g/L) | [H₂O]_{SLV}) |
| 0 | 10.23 | 2.37 | 3.34 | 41.5% |
| 10 | 10.04 | 3.64 | 4.22 | 46.3% |
| 20 | 8.88 | 4.54 | 5.11 | 47.1% |
| 30 | 8.05 | 5.05 | 5.31 | 48.7% |
| 40 | 6.72 | 6.28 | 5.46 | 53.5% |
| 50 | 5.46 | 6.38 | 5.78 | 52.5% |
| 60 | 4.91 | 7.60 | 6.29 | 54.7% |
| 70 | 3.43 | 8.09 | 6.38 | 55.9% |
| 80 | 3.42 | 8.35 | 6.27 | 57.1% |
| 90 | 2.75 | 8.63 | 6.05 | 58.8% |

### Example 19

### Continuous Butanol Fermentation and Membrane Solvent Extraction Process

1-Butanol or isobutanol are produced via fermentation using corn-derived or sugar cane-derived sugars or possibly other starch-based sugars. Butanol-producing microorganisms are native or engineered Clostridium acetobutylicum, Clostridium beijerinckii, yeast or E. Coli. Butanol is a powerful feedback inhibitor of the microorganisms that produce it. Butanol concentration of as low as 2 wt-% can shut down fermentation. When butanol is continuously extracted from the fermentation broth using membrane solvent extraction, this feedback inhibition by butanol is reduced, resulting in acceleration of the fermentation and butanol production rate. After extraction, the butanol and a slight amount of water is recovered by flash separation, vacuum distillation, or other downstream enrichment process. This results in lower overall energy of separation of butanol compared to separation by conventional distillation.

All patents and publications referred to herein are hereby incorporated by reference in their entirety. Various modifications and alterations of this invention may be made by those skilled in the art without departing from the scope and spirit of this invention, and it should be understood that this invention is not to be unduly limited to the illustrative embodiments set forth herein.

1. A method of producing butanol, the method comprising:
introducing an aqueous mixture comprising carbohydrates obtained from non-cellulosic biomass into a fermenter;
fermenting the aqueous mixture to provide a first fermentation broth, the fermentation broth comprising:
a microorganism for producing butanol;
carbohydrates from the non-cellulosic biomass; and
butanol; and
at least partially extracting the butanol from the first fermentation broth with a first solvent extractant by a first liquid-liquid extraction through a first porous membrane to provide a first extract and a second fermentation broth;
wherein the first solvent extractant comprises a straight-chain or branched alcohol having from 7 to 12 carbon atoms;
wherein the second fermentation broth has a lower concentration of the butanol than the first fermentation broth;
wherein the first liquid-liquid extraction is carried out in a liquid-liquid extraction element comprising:
a plurality of first layer pairs, each first layer pair comprising:
a first polymeric microporous membrane; and
a first flow channel layer oriented in a first flow direction having a first fluid inlet and a first fluid outlet disposed on first opposing sides of the liquid-liquid extraction element; and
a plurality of second layer pairs, with at least one second layer pair being disposed between two first layer pairs and at least one first layer pair being disposed between two second layer pairs so as to form a stack of layers, each second layer pair comprising:
a second polymeric microporous membrane; and
a second flow channel layer oriented in a second flow direction different than the first flow direction and having a second fluid inlet and a second fluid outlet disposed on second opposing sides of the extraction element.

2. The method of claim 1 wherein the butanol produced is 1-butanol.

3. The method of claim 1 further comprising directing the second fermentation broth back into the fermenter thereby increasing the rate of butanol production relative to a non-recycling method.

4. The method of claim 1 wherein the first extractant has a boiling point that is at least 30° C. higher than the butanol produced, or, if a mixture is produced, 30° C. higher than the higher boiling butanol produced.

5. The method of claim 1 wherein the first extractant comprises a straight-chain or branched alcohol having from 8 to 11 carbon atoms.

6. The method of claim 5 wherein the first extractant comprises a 2-octanol, 2-ethyl-1-hexanol, 1-nonanol, 2,6-dimethyl-4-heptanol, 1-decanol, 4-decanol, 2-propyl-1-heptanol, or combinations thereof.

7. The method of claim 1 wherein the microorganism for producing butanol comprises native or engineered Clostridium acetobutylicum, Clostridium beijerinckii, yeast, E. Coli, or a combination thereof.

8. The method of claim 1 wherein a portion of the first extractant becomes entrained in the second fermentation broth, the method further comprises at least partially extracting the entrained first extractant from the second fermentation broth with a second extractant by a second liquid-liquid extraction.

9. The method of claim 8 wherein the second extractant comprises dodecane.

10. The method of claim 1 wherein the non-cellulosic biomass comprises corn, sugar cane, sugar beets, cassava, wheat, or mixtures thereof.

11. A method of recovering butanol from a fermentation broth, the method comprising:
introducing an aqueous mixture comprising carbohydrates obtained from non-cellulosic biomass into a fermenter;
fermenting the aqueous mixture to provide a first fermentation broth, the fermentation broth comprising:
a microorganism for producing butanol;
carbohydrates from the non-cellulosic biomass; and
butanol;
at least partially extracting the butanol from the first fermentation broth with a first solvent extractant by a first liquid-liquid extraction through a first porous membrane to provide a first extract and a second fermentation broth; and
recovering at least a portion of the butanol from the first extract;
wherein the first solvent extractant comprises a straight-chain or branched alcohol having from 7 to 12 carbon atoms;
wherein the second fermentation broth has a lower concentration of the butanol than the first fermentation broth;
wherein the first liquid-liquid extraction is carried out in a liquid-liquid extraction element comprising:
a plurality of first layer pairs, each first layer pair comprising:
a first polymeric microporous membrane; and
a first flow channel layer oriented in a first flow direction having a first fluid inlet and a first fluid outlet disposed on first opposing sides of the liquid-liquid extraction element; and
a plurality of second layer pairs, with at least one second layer pair being disposed between two first layer pairs and at least one first layer pair being disposed between two second layer pairs so as to form a stack of layers, each second layer pair comprising:
a second polymeric microporous membrane; and
a second flow channel layer oriented in a second flow direction different than the first flow direction and having a second fluid inlet and a second fluid outlet disposed on second opposing sides of the extraction element.

12. The method of claim 11 wherein recovering the butanol comprises concentrating it by flash separation and/or vacuum distillation.

13. The method of claim 11 wherein the first extractant has a boiling point that is at least 30° C. higher than the butanol produced, or, if a mixture is produced, 30° C. higher than the higher boiling butanol produced.

14. The method of any one of claim 11 wherein the first extractant comprises a straight-chain or branched alcohol having from 8 to 11 carbon atoms.

15. The method of claim 14 wherein the first extractant comprises a 2-octanol, 2-ethyl-1-hexanol, 1-nonanol, 2,6-dimethyl-4-heptanol, 1-decanol, 4-decanol, 2-propyl-1-heptanol, or combinations thereof.

16. The method of claim 11 wherein the microorganism for producing butanol comprises native or engineered Clostridium acetobutylicum, Clostridium beijerinckii, yeast, E. Coli, or a combination thereof.

17. The method of claim 11 wherein a portion of the first extractant becomes entrained in the second fermentation broth, the method further comprises at least partially extracting the entrained first extractant from the second fermentation broth with a second extractant by a second liquid-liquid extraction.

18. The method of claim 17 wherein the second extractant comprises dodecane.

19. The method of claim 11 wherein the non-cellulosic biomass comprises corn, sugar cane, sugar beets, cassava, wheat, or mixtures thereof.

20. The method of claim 11 wherein the butanol produced is isobutanol.
